(19) United States
(12) Patent Application Publication
(10) Pub. No.: US 2022/0202410 A1
(43) Pub. Date: Jun. 30, 2022

(54) SYSTEMS, DEVICES, AND METHODS FOR BONE SUTURE ATTACHMENT AND SUPPORT

(71) Applicant: AESCLEPIUS CORPORATION, La Jolla, CA (US)

(72) Inventors: Adam H. Hsieh, La Jolla, CA (US); Joe Ty Lin, San Diego, CA (US)

(21) Appl. No.: 17/570,039

(22) Filed: Jan. 6, 2022

Related U.S. Application Data
(63) Continuation of application No. PCT/US20/41416, filed on Jul. 9, 2020.
(60) Provisional application No. 62/872,530, filed on Jul. 10, 2019.

(51) Int. Cl. A61B 17/04; A61L 31/02

(52) U.S. Cl. CPC A61B 17/0401; A61L 31/028; A61B 2017/0453; A61B 2017/0445; A61B 2017/0414

(57) ABSTRACT

Systems, devices, and methods are provided for attaching and supporting a bone suture. In particular, described herein are embodiments of implantable bracing apparatuses comprising one or more curved tubes configured to be implanted in one or more bone tunnels, and further configured to pass one or more sutures therethrough. Embodiments of methods of creating a bracing apparatus in situ by injecting a fluidic agent into a bone tunnel and inducing a phase transition are also described. Furthermore, described herein are embodiments of tunneling devices for creating a bone tunnel, which can be used with any of the bracing apparatuses described herein.

### CROSS-REFERENCE TO RELATED APPLICATIONS

This application is a continuation of International Patent Application No. PCT/US20/41416, filed Jul. 9, 2020, which claims priority to and the benefit of U.S. Provisional Patent Application No. 62/872,530 filed Jul. 10, 2019, both of which are incorporated by reference herein in their entireties for all purposes.

### FIELD

The subject matter described herein relates generally to systems, devices, and methods for attaching and supporting a suture to a bone. In particular, described herein are embodiments of bracing apparatuses for bone suture attachment and support, as well as methods and devices relating thereto.

### BACKGROUND

Joint arthropathies (diseases that compromise joint function) are part of a steadily growing worldwide trend in chronic musculoskeletal disorders. In 2012, the Bone and Joint Initiative published findings that one out of every two Americans were diagnosed with musculoskeletal conditions, accounting for hundreds of billions of dollars in costs, which continue to grow annually. In 2018, the World Health Organization (WHO) identified the second largest contributor to global disability as musculoskeletal conditions. The increasing number of afflicted people and a continued rise in treatment costs point to a critical need for new technologies that provide more effective solutions to manage musculoskeletal ailments.

Joint arthropathies caused by soft tissue damage (e.g., tendon, ligament, and/or fibrocartilage tears) make up the majority of cases within the broader category of musculoskeletal conditions. Shoulder pain stands among the most common musculoskeletal complaint worldwide, with rotator cuff tears being the leading cause of shoulder disability. Other types of ligament, tendon, and fibrocartilage injuries, such as labral tears, meniscus root tears, Achilles tendon avulsions, anterior cruciate ligament (ACL) ruptures, and lateral ankle ligament tears, among others, are somewhat less prevalent, but no less debilitating. Most of these injuries, whether due to tear size or lack of responsiveness to conservative treatment (e.g. physical therapy), require primary surgical repair. In 2014, the United States Agency for Healthcare Research and Quality (AHRQ) reported over 1.8 million invasive, therapeutic surgeries involving “muscle, tendon, soft tissue operating room procedures” and “incision or fusion of joint, or destruction of joint lesion” in the United States, which equates to 8.3% of the roughly 21.7 million total ambulatory and inpatient surgical procedures.

The goal of such repairs is to re-establish the position and direction of force transmission in these tissues, in order to restore stability and motion to their respective joints. For soft tissue injuries, this can be achieved by re-attaching the torn areas of soft tissue (e.g., tendon, ligament, and/or fibrocartilage)—which naturally pulls away from its anatomic insertion site upon injury—using a fixation method to create a stable connection and close contact between tissue and bone so that the interface can heal over time. The fixation method should be mechanically and structurally robust, because the biomechanical forces generated by muscles and joint motion may reach several hundred Newtons during physiological function. The fixation method should also be sufficient to withstand thousands of cycles of repetitive loading, particularly in the lower extremities.

Due to anatomical and functional variation, techniques used to achieve soft tissue repair can depend on the particular application. Two approaches for soft tissue reattachment, for example, include: (1) transosseous repair and (2) suture anchored repairs.

In a transosseous approach, a bone tunnel is created at a location corresponding to the injured tissue. A remaining portion of the torn tissue, a synthetic/autologous/cadaveric tissue graft, or a suture tethered to the injured tissue can be passed through the bone tunnel and either tied back around the surface of the bone or affixed by an interference screw or button device. This approach, is traditionally performed as open surgery, which may require a large incision to give the surgeon access to the bone and joint, or more recently with arthroscopic surgical tools.

Due to anatomical limitations, a transosseous approach for rotator cuff repairs involves passing sutures through multiple tunnels that intersect at a location within bone. At this intersection, angular features place sutures at risk for abrasive failure. Moreover, even in the absence of sharp angles, sutures can cut through bone unpredictably, for example, when the sutures are being tensioned during surgery. In certain cases, a surgeon can insert a cortical reinforcement, such as a polymer grommet, into the entrance of the bone tunnel to reduce failure risk. However, this method can be disadvantageous for various reasons, including requiring the subjective judgment of the surgeon, and can result in a high variability in outcomes. Moreover, there are constraints in implementing multiple bone tunnels because of the long traverse required.

Suture anchored repair is another approach to repair rotator cuff tendons, as well as the labrum, meniscus root, lateral ankle ligaments, and others. With respect to suture anchored repair, a metal or polymer suture anchor is secured by way of screw or interference fit into a pilot hole created in the bone. Sutures are tethered to the anchor and are used to tie the tissue back to its anatomic insertion site, thereby restoring function. In some cases, the anchor can include suture knots and/or deployable securing elements. In other cases, the anchor can be an all-suture soft anchor comprising a polymer textile sleeve through which a suture runs. Once inserted into the pilot hole, the sleeve bunches together when the suture line is pulled, creating a plug that is slightly wider than the pilot hole, to hold the suture in place.

Suture anchored repair relies on the interface between the anchor and the bone to maintain structural integrity of the repair. This can be disadvantageous in that the anchors lack the ability to achieve full biological integration, such that with time there will be a risk of failure if tissue healing remains inadequate. Even with a secure suture anchor, the rate of re-tear in a rotator cuff repair has been reported to be between 30% and 70%. Furthermore, the size and placement of anchors limit the sutures that can be used for repair. For instance, if there are complications or failures in a primary repair where a suture anchor is used, surgeons are faced with the dilemma of having constraints on anchor placement for the secondary repair.

Thus, needs exist for systems, devices and methods that are more mechanically and structurally robust for attaching and supporting a bone suture.

### SUMMARY

Provided herein are example embodiments of systems, devices and methods for attaching and supporting a suture to a bone. Generally, an implantable bracing apparatus comprising one or more curved tubes are described, wherein the one or more curved tubes are configured to be implanted within one or more bone tunnels.

In some example embodiments, for example, the implantable bracing apparatus can comprise a curved tube at least a portion of which is configured to be implanted within a bone tunnel, wherein the curved tube includes an outer surface configured to interface with the bone tunnel, an inner lumen configured to pass a suture therethrough and to prevent the suture from contacting at least a portion of the bone tunnel, a first open end corresponding with a first entry point of the bone tunnel, and a second open end corresponding with a second entry point of the bone tunnel.

In other example embodiments, an implantable bracing apparatus can include a plurality of curved tubes, wherein at least a portion of each of the plurality of curved tubes is configured to be implanted within a plurality of bone tunnels, and wherein a first curved tube of the plurality of curved tubes comprises an outer surface configured to interface with a first bone tunnel, an inner lumen configured to pass a suture therethrough and to prevent the suture from contacting at least a portion of the first bone tunnel, a first open end corresponding with a first entry point of the first bone tunnel, and a second open end corresponding with a second entry point of the first bone tunnel. In some embodiments, a second curved tube intersects with the first curved tube through a set of apertures, wherein the second curved tube is configured to pass a second suture therethrough. In other embodiments, the second curved tube can be solid, positioned adjacent to the first curved tube without intersecting it, and provided to passively support the first curved tube by distributing stress generated by the suture passing through the first curved tube. In other embodiments, the bracing apparatus can comprise three or four curved tubes.

In some of the example embodiments, an implantable bracing apparatus comprises a cylindrical volume comprising one or more helices (e.g., a single helical coil, a double helical coil, etc.) or a latticework of struts.

In other example embodiments, a method of generating a bracing apparatus in situ is described, wherein the method comprises: introducing at least a portion of an injector into a bone tunnel, wherein the injector includes a plurality of perforations or holes; injecting a fluidic agent through the plurality of perforations or holes into the bone tunnel; causing the fluidic agent to undergo a phase transition from a liquid state to a substantially solid state, wherein the substantially solid state of the fluidic agent comprises a bracing apparatus; and retracing the injector from the bone tunnel. In some embodiments, the phase transition can be caused, for example, by use of LEDs of a visible, ultraviolet, or infrared wavelength, thermal or electrical conductors, or chemical diffusion agents. According to some embodiments, the fluidic agent can comprise a self-assembling polymer, a UV-cured resin, a thermosetting polymer, a chemically-cured material, or a chemically-crosslinked material.

In addition, example embodiments of tunneling devices are described herein, wherein the tunneling devices can be used with any of the bracing apparatuses also described herein. In some example embodiments, a tunneling device for creating a bone tunnel can comprise: a housing comprising at least one surface configured to interface with a bone material; a channel disposed within the housing; an impactor configured to travel along the channel, wherein the impactor comprises one or more pointed ends configured to strike the bone material; an internal propulsion mechanism configured to cause the impactor to travel along the channel in a back-and-forth motion, and to cause the one or more pointed ends of the impactor to strike the bone material. In some example embodiments, the internal propulsion mechanism can comprise a piezoelectric motor including a plurality of piezoceramic elements and a plurality of bumpers disposed along the channel. According to some embodiments, the internal propulsion mechanism can be configured to cause a first pointed tip at a first end of the impactor to strike the bone material at a first entry point. According to other embodiments, the internal propulsion can also be configured to cause a second pointed tip at a second end of the impactor to strike the bone material at a second entry point. Furthermore, in some embodiments, the impactor of the tunneling device can comprise a hollow cylinder configured to contain and release an implantable bracing apparatus, such as those described throughout the present disclosure.

The various configurations of these systems, methods and devices are described by way of the embodiments which are only examples. Other systems, devices, methods, features, improvements and advantages of the subject matter described herein are or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIGS. 1A and 1B are a perspective view and a cross-sectional view of an example embodiment of an implantable bracing apparatus.

FIG. 1C is a perspective view of an example embodiment of an implantable bracing apparatus with a flange.

FIGS. 1D to 1G are perspective and side views of example embodiments of end cap accessories.

FIGS. 1H and 1I are progressive side views of an example embodiment of a ridge locking feature.

FIG. 1J is a partial cross-sectional view of an example embodiment of an implantable bracing apparatus.

FIGS. 2A and 2B are a perspective view and a cross-sectional view of another example embodiment of an implantable bracing apparatus.

FIGS. 3A and 3B are a perspective view and an overhead cross-sectional view of another example embodiment of an implantable bracing apparatus.

FIG. 3C is a partial cross-sectional view of another example embodiment of an implantable bracing apparatus.

FIGS. 4A and 4B are a perspective view and a partial side view of another example embodiment of an implantable bracing apparatus.

FIGS. 5A and 5B are a perspective view and an overhead view of another example embodiment of an implantable bracing apparatus.

FIGS. 6A and 6B are a perspective view and an overhead view of another example embodiment of an implantable bracing apparatus.

FIGS. 7A and 7B are a perspective view and a side view of another example embodiment of an implantable bracing apparatus.

FIGS. 8 and 9 are perspective views of example embodiments of implantable bracing apparatuses.

FIGS. 10A and 10B are a side view and a perspective view of another example embodiment of an implantable bracing apparatus.

FIGS. 11A to 11D are perspective views and a side view of other example embodiments of implantable bracing apparatuses.

FIGS. 12A and 12B are a top view and perspective view of a curved tubular injector.

FIGS. 13A to 13F are partial cross-sectional views of curve tubular injector at various stages of operation.

FIG. 14 is a flow diagram of an example embodiment of a method for generating a bracing apparatus.

FIGS. 15A to 15C are perspective views of an example embodiment of a tunneling device.

FIGS. 16A and 16B are perspective views of another example embodiment of a tunneling device.

FIGS. 17A to 17D are progressive diagrammatic views of an example embodiment of a tunneling device in various stages of operation.

FIGS. 18A to 18F are progressive diagrammatic views of another example embodiment of a tunneling device in various stages of operation.

### DETAILED DESCRIPTION

Before the present subject matter is described in detail, it is to be understood that this disclosure is not limited to the particular embodiments described herein, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for attaching and supporting a bone suture. Accordingly, some embodiments include implantable bracing apparatuses to reinforce a hole or tunnel in a bone. These various embodiments can include elements through which sutures pass and/or to which sutures may be tethered. In certain embodiments, some or all elements of the bracing apparatus may comprise metal, natural or synthetic material, organic or inorganic material, biodegradable or non-biodegradable polymer, or a combination thereof.

In some embodiments, a bracing apparatus can be a flexible or rigid solid-walled curved tube that is inserted into a pre-formed bone tunnel. The bracing apparatus can further comprise ends having one or more attachment features to which accessories can be attached, depending on the specific surgical application.

In other embodiments, the bracing apparatus can be a flexible or rigid curved tube having a non-solid wall. In some embodiments, for example, a bracing apparatus can comprise a coil having single or multiple helices, wherein the single or multiple helices are configured to compress and facilitate the insertion of the bracing apparatus. In other embodiments, a bracing apparatus can comprise a simple or complex latticework of struts, wherein the latticework of struts can be collapsible into a narrow configuration and, further, configured to expand during deployment while inside a bone tunnel.

According to another aspect of the embodiments, a method for creating a bracing apparatus in situ using phase transition polymers is provided. In some embodiments, a liquid polymer can be extruded from perforations in the wall of a straight or curved injector. Upon extrusion, the polymer can undergo a phase transition to a solid, where the phase transition can be induced by temperature, chemical or enzyme crosslinking/curing, photoreactive crosslinking using ultraviolet, infrared, or visible light, or other means. Following the phase transition, the injector is withdrawn, leaving the bracing apparatus within the bone tunnel or hole.

According to another aspect of the embodiments, a tunneling device is provided for creating a curved bone tunnel and inserting a bracing apparatus into the bone tunnel. In certain embodiments, the tunneling device can include a curved channel or guide tube configured to guide a pointed impactor along a predetermined path. In some embodiments, for example, one or more ends of the curved track or guide tube abut a target area of a bone surface, wherein the target area comprises one or more predetermined entry and exit points of a tunnel to be created in the bone. In some embodiments, a tunneling device can also include a means for propelling the impactor, for example, through the use of a pneumatic, magnetic, electrical, or mechanical mechanism, or a combination thereof.

For each and every embodiment of a method disclosed herein, systems and devices capable of performing each of those embodiments are covered within the scope of the present disclosure. For example, embodiments of tunneling devices for creating a bone tunnel are disclosed, and these devices can each have one or more internal propulsion mechanisms, piezo motors, piezoceramic elements, bumpers, suture feed mechanisms, and other components that can perform any and all method steps, or facilitate the execution of any and all method steps.

### Example Embodiments of Implantable Bracing Apparatuses

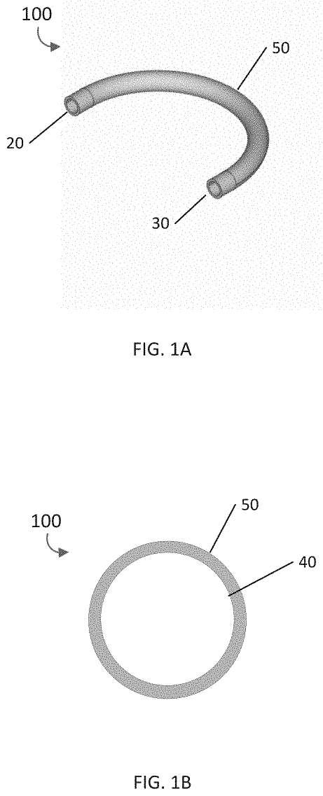

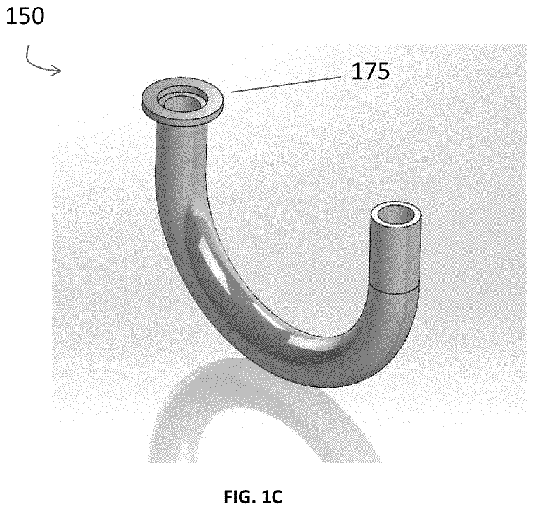

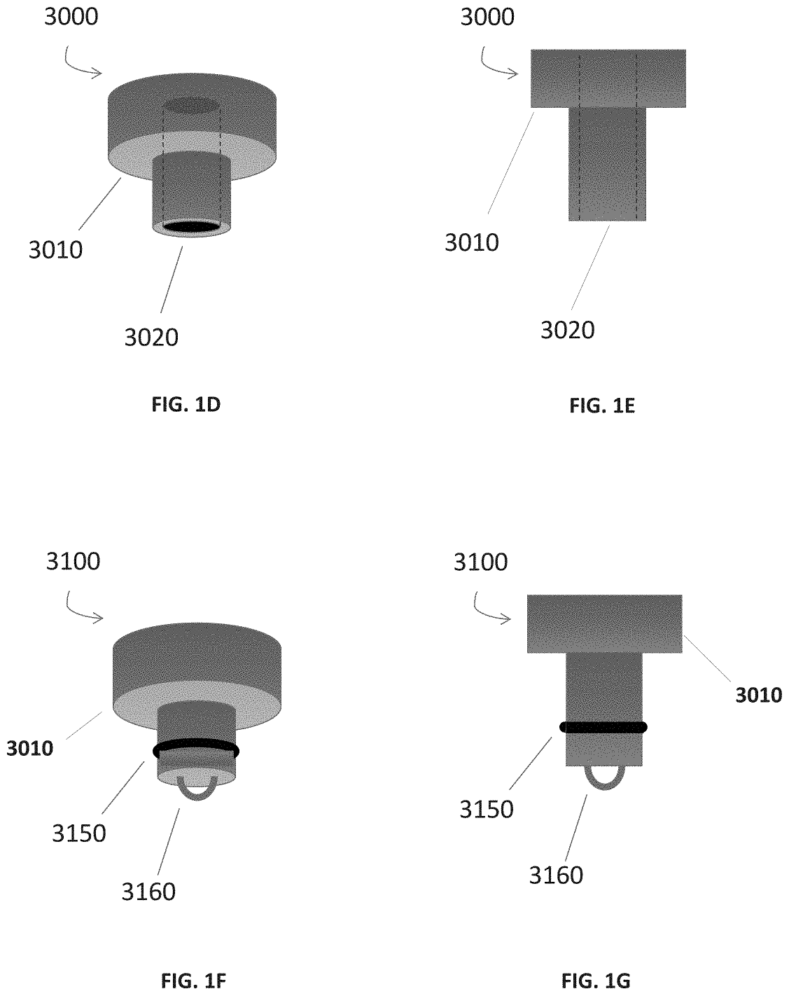

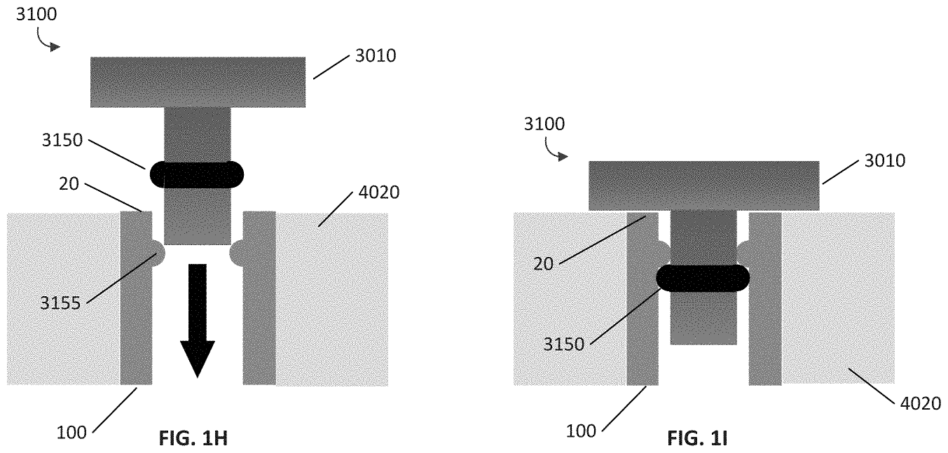

FIGS. 1A and 1B depict a perspective view and a cross-sectional view, respectively, of an example embodiment of an implantable bracing apparatus 100. According to one aspect of the embodiment, apparatus 100 can comprise a curved tube at least a portion of which is configured to be implanted within a bone tunnel, wherein the curved tube includes an inner lumen 40, an outer surface 50, and two open ends 20, 30. Those of skill in the art will appreciate that the curved tube can have regional variations in radius of curvature along the length of the tube, including areas that are straight. Indeed, for certain applications, the entire length of tube that serves as the bracing apparatus may be straight. According to one aspect of the embodiments, outer surface 50 is configured to interface with the bone tunnel, and inner lumen 40 is configured to pass a suture therethrough and to prevent the suture from contacting at least a portion of the bone tunnel. According to another aspect of the embodiments, first open end 20 of apparatus 100 can correspond with a first entry point of the bone tunnel, and second open end 30 can correspond with a second entry point of the bone tunnel.

As best seen in FIG. 1B, apparatus 100 can have a circular cross-sectional geometry. Those of skill in the art, however, will appreciate that the cross-sectional geometry of bracing apparatus 100 can be non-circular (e.g., oblong), and may have other geometric features that provide functional or structural utility. For example, in some embodiments, grooves, fins, or flanges integral to the apparatus can be disposed on outer surface 50 and/or open ends 20, 30. In example embodiment FIG. 1C, apparatus 150 is shown with one of the open ends 175 having an integral flange, which constrains the apparatus from further entering the bone tunnel. Furthermore, some or all elements of bracing apparatus 100 can comprise a metallic material, natural or synthetic material, organic or inorganic material, biodegradable or non-biodegradable polymer, or a combination thereof. According to certain embodiments, for example, inner lumen 40 can comprise a coating of polyethylene or polytetrafluoroethylene composites to reduce friction. According to other embodiments, osteoconductive materials, such as hydroxyapatite, can be incorporated into inner lumen 40 and/or outer surface 50 to enhance osseointegration and/or bone ingrowth. Proteins, other biologics, or synthetic molecules can also be tethered to either inner lumen 40 or outer surface 50 to achieve the same or similar results. Further, some or all elements of bracing apparatus 100 can be subject to surface modification to enhance osseointegration, such as, for example, plasma treatment or electrochemical etching to generate nanotextured surfaces.

In some embodiments, one or both open ends 20, 30 of bracing apparatus 100 can be configured to receive and mate with one or more end cap accessories. FIGS. 1D to 1G depict perspective and side views of two example embodiments of end cap accessories 3000 and 3100, respectively. As shown in FIGS. 1D and 1E, end cap accessory 3000 features a flanged head 3010 and a through hole 3020 to enable passage of a suture. The shaft of end cap accessory 3000 can be configured to mate with open ends 20, 30 of bracing apparatus 100, for example, either by press fit or snap fit via rounded ridges.

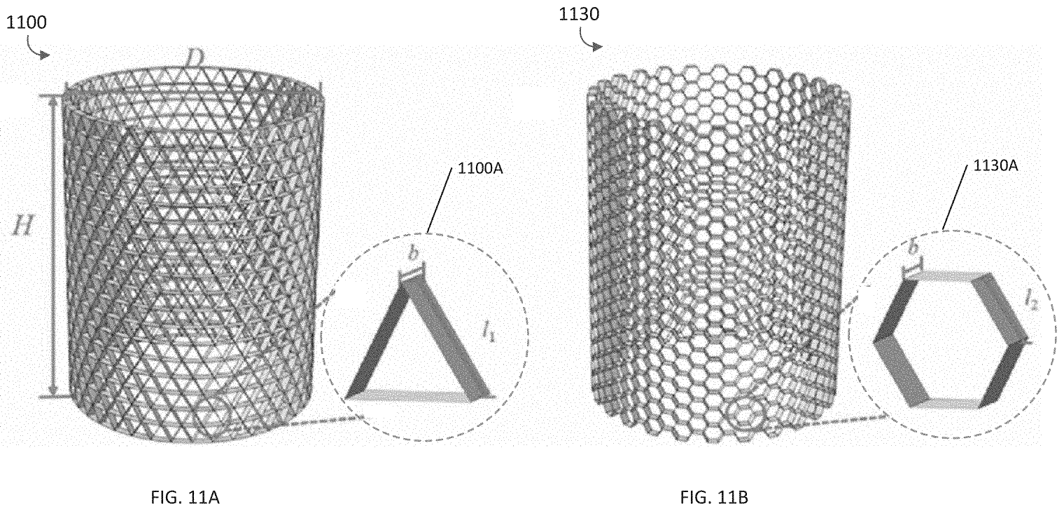

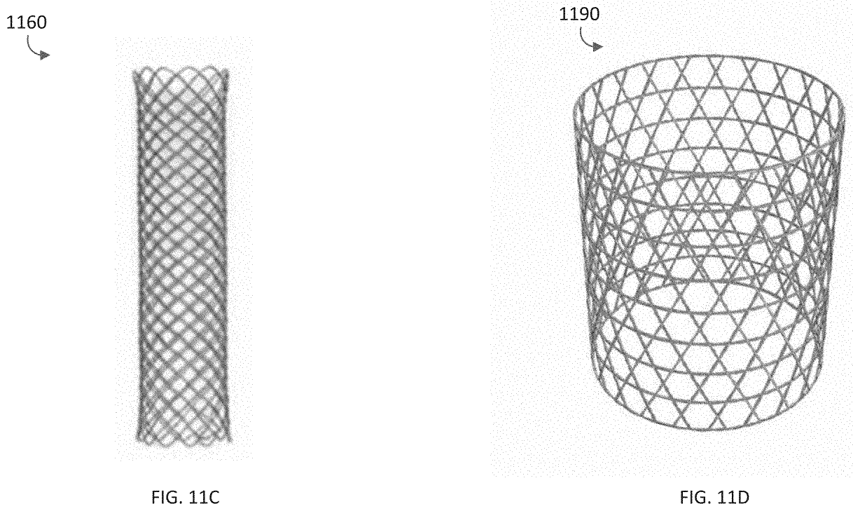

FIGS. 1F and 1G depict another embodiment of an end cap accessory 3100 with flanged head 3010, and which also includes an eyelet 3160 at the end of the shaft. According to another aspect of the embodiments, a ridged feature 3150 disposed along the shaft is configured to lock end cap accessory 3100 with a corresponding ridge locking feature 3155 (shown in FIGS. 1H and 11) at the ends 20, 30 of bracing apparatus 100.

FIGS. 1H and 1I depict progressive side views of the ridge locking feature in operation, as described with respect to the end cap accessory 3100 (shown in FIGS. 1F and 1G). According to some embodiments, each of the one or more end cap accessories 3100 can include a ridged feature 3150 along an outer diameter of the shaft. As end cap accessory 3100 is moved in a downward direction, as indicated by the downward arrow, ridged feature 3150 engages, by snap-fit or press-fit mating, a corresponding ridge locking feature 3155 disposed within end 20 of bracing apparatus 100, which is shown deployed within bone 4020. Further, as best seen in FIG. 11, when the ridge locking feature is engaged, a lower surface of flanged head 3010 of end cap accessory 3100 abuts the top surface of open end 20 of bracing apparatus 100.

FIG. 1J is a partial cross-sectional view depicting implantable bracing apparatus 100 with two end cap accessories 3000, as deployed within bone 4020. According to the depicted embodiment, an end cap accessory 3000 is disposed at each of open ends 20, 30 of bracing apparatus 100. Soft tissue 4010, which is configured to be to be re-attached, is situated next to bone 4020. Suture 4050 passes through the soft tissue 4010, enters a first through hole 3020 of a first end cap accessory 3000, traverses one end 20 of bracing apparatus 100 to the other open end 30, exits a second through hole 3020 of a second end cap accessory 3000, and finally traverses soft tissue 4010.

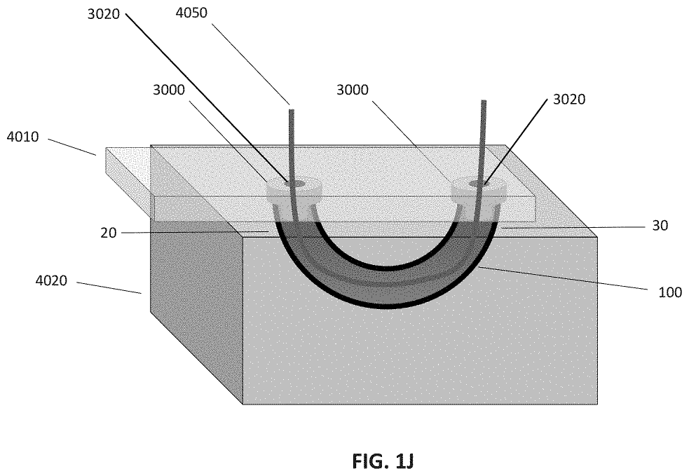

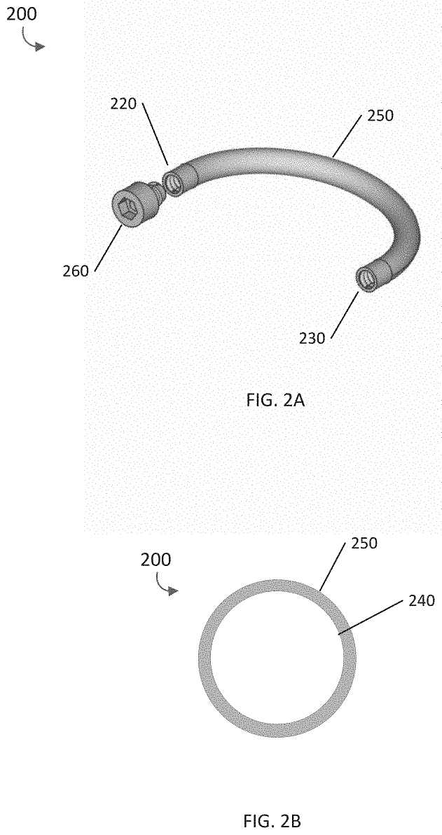

FIGS. 2A and 2B depict a perspective view and a cross-sectional view, respectively, of another example embodiment of an implantable bracing apparatus 200. Similar to the embodiments described with respect to FIG. 1A, bracing apparatus 200 can comprise a curved tube at least a portion of which is configured to be implanted within a bone tunnel, wherein the curved tube includes an inner lumen 240, an outer surface 250, and two open ends 220, 230. Although bracing apparatus 200 is depicted in FIG. 2B as having a circular cross-section, those of skill in the art will appreciate that the cross-sectional geometry of apparatus 200 can be non-circular (e.g., oblong), and may have other geometric features that provide functional or structural utility (e.g. grooves or fins on the outer surface or ends). Furthermore, some or all elements of bracing apparatus 200 can comprise a metallic material, natural or synthetic material, organic or inorganic material, biodegradable or non-biodegradable polymer, or a combination thereof. According to certain embodiments, for example, inner lumen 240 can comprise a coating of polyethylene or polytetrafluoroethylene composites to reduce friction. According to other embodiments, osteoconductive materials, such as hydroxyapatite, can be incorporated into inner lumen 240 and/or outer surface 250 to enhance osseointegration and/or bone ingrowth. Proteins, other biologics, or synthetic molecules can also be tethered to either inner lumen 240 or outer surface 250 to achieve the same or similar results. Further, some or all elements of bracing apparatus 200 can be subject to surface modification to enhance osseointegration, such as, for example, plasma treatment or electrochemical etching to generate nanotextured surfaces.

In some embodiments, one or both open ends 220, 230 of bracing apparatus 200 can include a threaded portion configured to receive and couple with a functional accessory, such as screw cap 260. Although FIG. 2A depicts the threaded portion disposed on an inner surface of lumen 240, those of skill in the art will appreciate that the threaded end portion can also be disposed on outer surface 250 of bracing apparatus 200. Each threaded end portion can be configured to receive one or more of a screw cap 260, flanged end cap (e.g., as described with respect to FIGS. 1C to 1D), anchor plate, or any other functional accessory to be mounted on one or both of ends 220, 230.

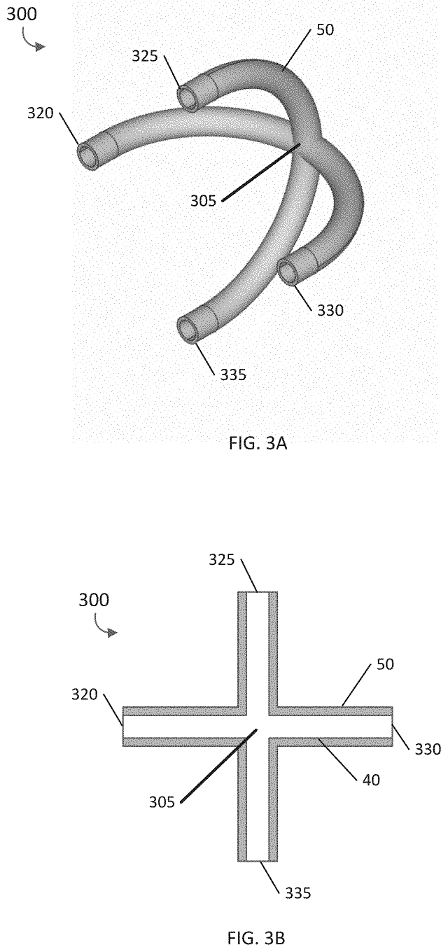

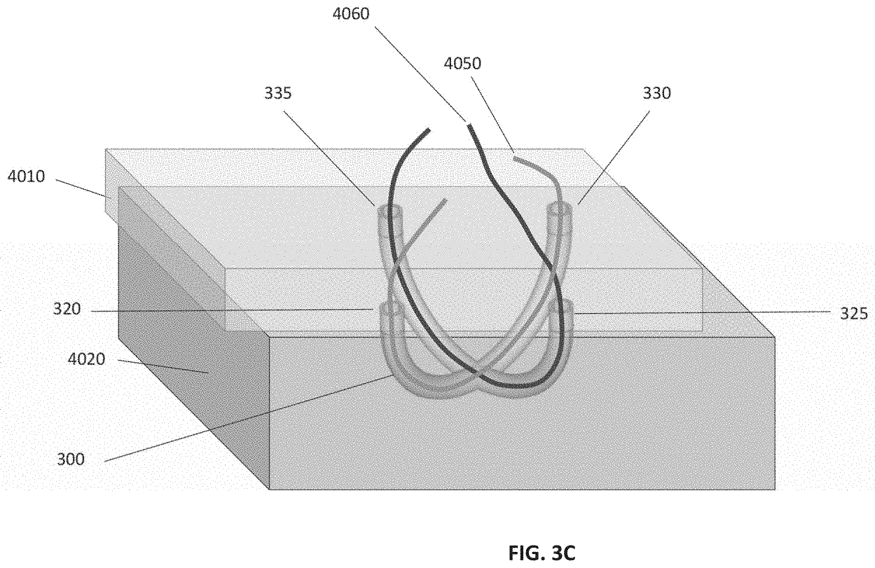

FIGS. 3A and 3B depict a perspective view and an overhead cross-sectional view, respectively, of another example embodiment of an implantable bracing apparatus 300 comprising a plurality of curved tubes, wherein at least a portion of each curved tube is configured to be implanted within a bone tunnel. According to one aspect of the embodiments, bracing apparatus 300 can include two curved tubes configured to intersect at a middle portion 305 along the length of each curved tube. In some embodiments, the curved tubes can be similarly dimensioned, e.g., having a similar diameter, wall thickness, etc. Apparatus 300 further includes two pairs of open ends (320, 325, 330, 335), each pair corresponding to a curved tube. As can be seen in FIG. 3B, apparatus 300 includes an inner lumen 40 and an outer surface 50. Although a typical arrangement is for the two intersecting segments to be orthogonal to one another, those of skill in the art will appreciate that the intersecting segments may be at any angle.

FIG. 3C depicts bracing apparatus 300 in a deployed state. Although FIG. 3C depicts bracing apparatus 300 without end cap accessories, those of skill in the art will appreciate that other embodiments of bracing apparatus 300 can be implemented with end cap accessories, such as those described with respect to FIGS. 1C to 1F. Referring back to FIG. 3C, soft tissue 4010 to be re-attached is adjacent to bone 4020, with first and second sutures 4050 and 4060 passing through soft tissue 4010. First suture 4050 traverses soft tissue 4010, enters open end 320 of bracing apparatus 300, exits from open end 330, then passes again through soft tissue 4010. Similarly, second suture 4060 traverses soft tissue 4010, enters open end 325 of an orthogonal segment of bracing apparatus 300, exits from the open end 335, then passes again through soft tissue 4010. Although FIG. 3C depicts bracing apparatus 300 implemented with two sutures, those of skill in the art will understand that a single suture can be used. For example, in some embodiments, suture 4050 enters end 320 and exits from end 330, wherein the bracing segment comprising ends 325, 335 is configured to serve as a passive support by distributing stress generated by suture 4050.

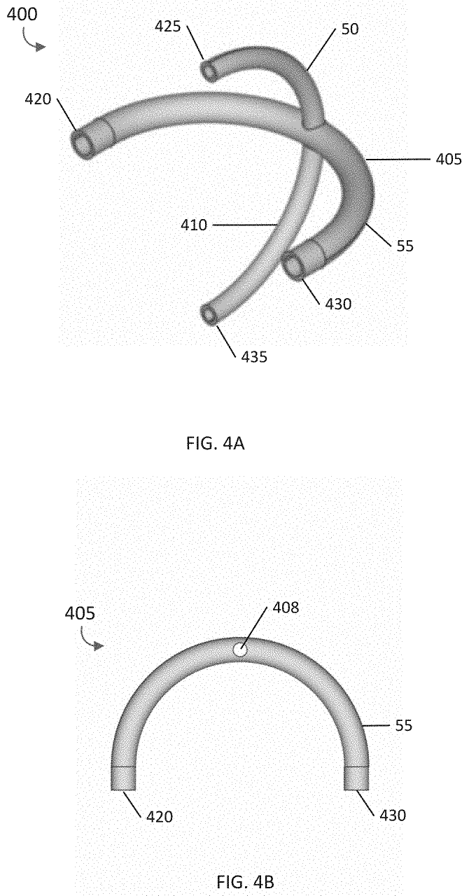

FIG. 4A is a perspective view of another example embodiment of an implantable bracing apparatus 400. According to one aspect of the embodiments, bracing apparatus 400 can comprise two intersecting curved tubes (405, 410), wherein the tubes are dissimilar. As can be seen in FIG. 4B, according to some embodiments, first curved tube 405 can include a first pair of centrally-located apertures 408. A second curved tube 410, which can have a smaller diameter relative to first curved tube 405, can be deployed by passing through apertures 408. In addition, according to some embodiments, second curved tube 410 can include a second pair of centrally-located apertures (not shown) configured to allow sutures to be passed through first curved tube 405. Second curved tube 410 can also include one or more raised features to ensure proper alignment of the first and second pairs of apertures of the first and second curved tube, respectively. As best seen in FIG. 4B, the first pair of apertures 408 of first curved tube 405 can be disposed along a middle portion of first curved tube 405.

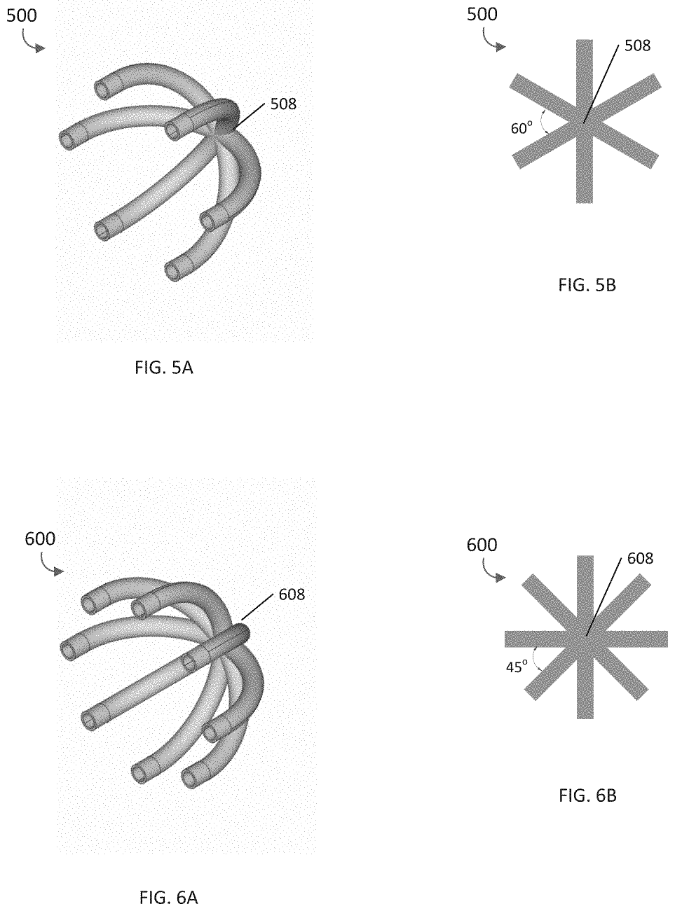

FIGS. 5A and 5B depict a perspective view and an overhead view, respectively, of another example embodiment of an implantable bracing apparatus 500. Similar to bracing apparatus 300, the depicted embodiments comprise a plurality of curved tubes of equal diameter that intersect orthogonally at an apex 508 of the curved tubes. With respect to bracing apparatus 500, there can be three curved tubes that intersect at an approximately 60 degree angle to each other at apex 508, as best seen in FIG. 5B. Bracing apparatus 500 can be deployed in a plurality of bone tunnels according to a process similar to that of bracing apparatus 300 (as described with respect to FIG. 3C), and further provides for the implementation of one, two, or three sutures. Those of skill in the art would also appreciate that the angles of intersection between the curved tubes can be greater or less than 60 degrees.

FIGS. 6A and 6B depict a perspective view and an overhead view, respectively, of another example embodiment of an implantable bracing apparatus 600. According to some embodiments, bracing apparatus 600 can comprise four curved tubes that intersect at an apex 608, wherein each curved tube is at a 45 degree angle to at least one adjacent curved tube. As with the previously described embodiments, bracing apparatus 600 can be deployed in a plurality of bone tunnels according to a process similar to that of bracing apparatus 300 (as described with respect to FIG. 3C), and further provides for the implementation of up to four sutures. Those of skill in the art will also appreciate that the angles of intersection between the curved tubes can be greater or less than 45 degrees.

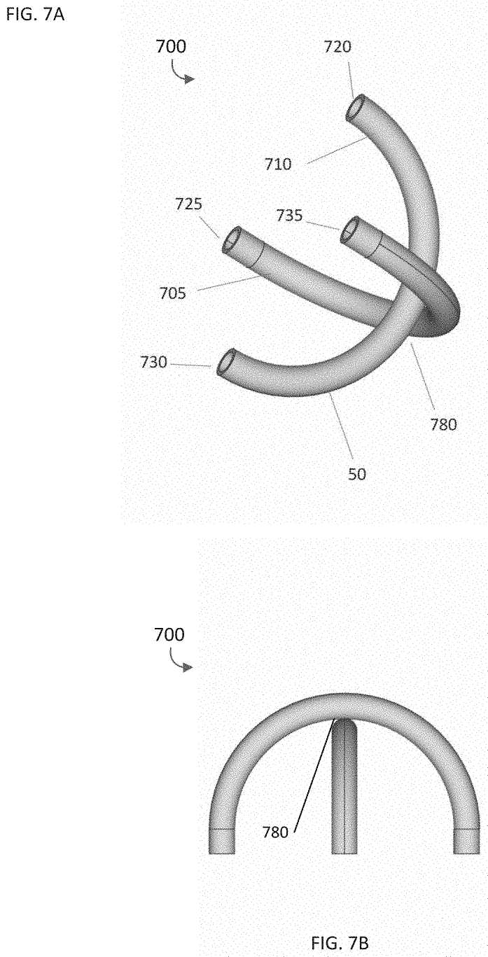

FIGS. 7A and 7B depict a perspective view and a side view, respectively, of another example embodiment of a bracing apparatus 700, comprising a first curved tube 705 and a second curved tube 710. As can be seen in the figures, bracing apparatus 700 can be configured to be deployed within two curved bone tunnels, wherein the first curved tube 705 is deployed within a first bone tunnel having a first depth within the bone, wherein the second curved tube 710 is deployed within a second bone tunnel having a second depth within the bone, and wherein the first depth is different from the second depth. In some embodiments, the first depth is greater than the second depth, and consequently, first curved tube 705 is deployed at a greater depth within the bone relative to second curved tube 710. According to another aspect of the embodiments, first curved tube 705 and second curved tube 710 “cross” at location 780, at an angle relative to one another. Although FIGS. 7A and 7B show the cross location 780 as an orthogonal angle, those of skill in the art will appreciate that the angle may be greater than or less than 90 degrees. Each of first curved tube 705 and second curved tube 710 may also have inner lumens (not shown) and outer surfaces 50, wherein the inner lumens are configured to pass one or more sutures. According to other embodiments, second curved tube 710 can be solid (e.g., without a lumen), and configured to serve as a passive support for first curved tube 705.

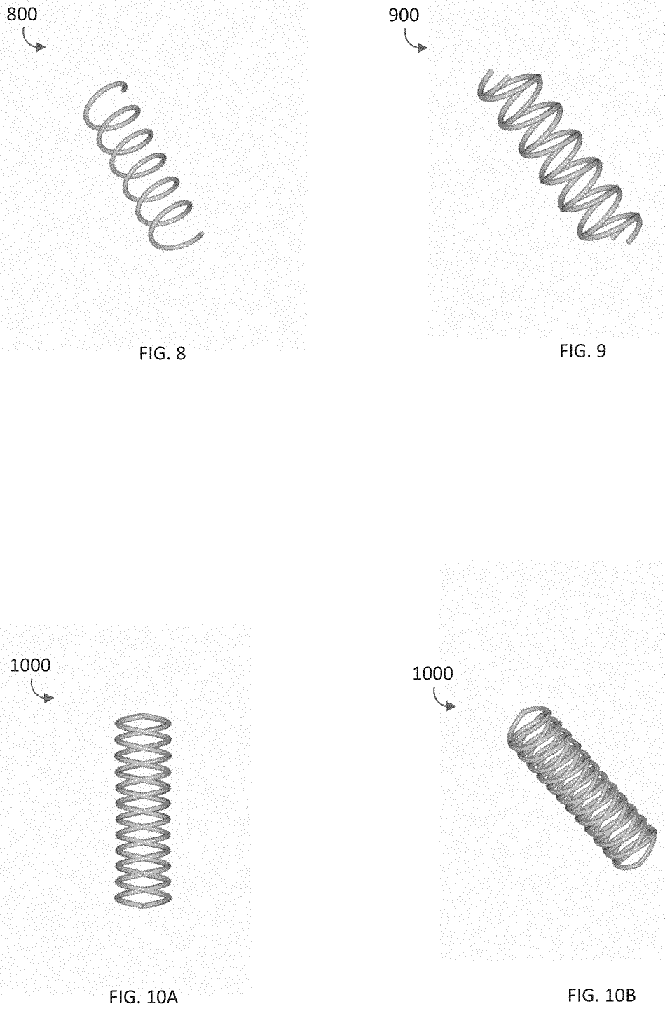

According to some embodiments, bracing apparatuses may also be manufactured to have flexibility, such as tubular structures with corrugated walls or with non-solid walls, e.g., bracing apparatuses that encompass a cylindrical volume. FIG. 8 is a perspective view of another example embodiment of implantable bracing apparatus 800 comprising a cylindrical volume comprising a single helical coil. Those of skill in the art will appreciate that the helical coil may possess different chirality, pitch, radius of curvature, slant angle, wire diameter, wire material, and wire cross-sectional geometry. Furthermore, helical coil bracing apparatuses may also possess spatial variations of different parameters within a single embodiment. The helical coil bracing apparatus can be inserted into bone tunnels that have regional variations in radius of curvature, as well as regions that are straight. FIG. 9 is a perspective view of an embodiment of implantable bracing apparatus 900 comprising a cylindrical volume comprising a double helix, which—in a manner similar to that of apparatus 800—may possess different physical and material characteristics, as well as spatial variations. FIGS. 10A and 10B depict a side view and a perspective view, respectively, of another example embodiment of implantable bracing apparatus 1000, wherein bracing apparatus 1000 comprises a cylindrical volume comprising four helical coils (e.g., two right-hand and two left-hand coils). In a similar manner, those of skill in the art will appreciate that corrugated bracing apparatuses may possess either concentric ring patterns of ridges and valleys along the length of the apparatus, or helical patterns of ridges and valleys that vary in helix configurations just as those described for the helical coil apparatuses above.

According to other embodiments, bracing apparatuses may also comprise a cylindrical volume comprising a latticework of struts, such as those depicted in FIGS. 11A to 11D. FIG. 11A shows a perspective view of an example embodiment of an implantable bracing apparatus 1100, wherein a lattice of triangular cells, as shown in exploded view 1100A, is used to form a cylindrical bracing structure. Each cell can have a thickness, b, and each side of triangular cell can have a length, l₁. Those of skill in the art will appreciate that the sides of the triangular cell can have similar or dissimilar lengths. FIG. 11B shows a perspective view of another example embodiment of an implantable bracing apparatus 1130, wherein a lattice of hexagonal cells, as shown in exploded view 1130A, is used to form a cylindrical bracing structure. Each cell can have a thickness, b, and each side of hexagonal cell can have a length, l₁. Those of skill in the art will appreciate that the sides of the hexagonal cell can have similar or dissimilar lengths. FIG. 11C shows a side view of yet another example embodiment of an implantable bracing apparatus 1160, where the apparatus includes a lattice comprising quadrilateral cells. FIG. 11D shows a perspective view of an example embodiment of an implantable bracing apparatus 1190, wherein the apparatus includes a lattice comprising multiple geometries (e.g., triangular and hexagonal). These embodiments of the latticework are intended to be illustrative only and are not meant to limit the scope of the present disclosure. Indeed, those of skill in the art will recognize that latticework can be constructed from one or more different materials, according to different geometries, in order to achieve the intended result of facilitating the insertion of the implantable bracing apparatus. The latticework bracing apparatus can also be inserted into bone tunnels that have regional variations in radius of curvature, as well as regions that are straight.

According to some embodiments, a bracing apparatus may be generated in situ (at the site of implantation). In particular, a curved tubular injector tip of an instrument can be introduced into a bone tunnel, and a natural or synthetic fluidic agent can be pumped through the instrument into the bone tunnel, where the agent subsequently undergoes a phase transition into a solid. The injector can be withdrawn from the bone tunnel after the phase transition, leaving behind a solid implanted bracing apparatus inside the bone tunnel.

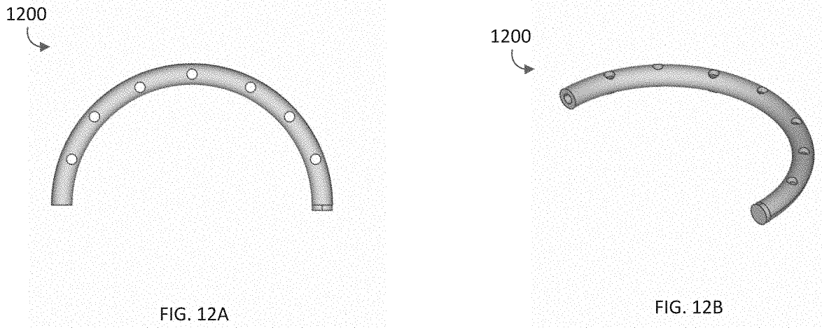

FIGS. 12A and 12B depict a top view and a perspective view, respectively, of an example embodiment of a curved tubular injector 1200 configured to be inserted into a bone tunnel and introduce a fluidic agent therein. As can be seen in the figures, curved tubular injector comprises a plurality of perforations. Those of skill in the art will appreciate that the number, size, pattern, and spacing of perforations in curved tubular injector 1200, as well as the material composition, size, cross-sectional geometry, length, and shape of the tube may vary to achieve the intended function, with portions of the curved tubular injector having variations in radius of curvature and/or regions that are straight. The length and shape of the curved tubular injector 1200 is dimensioned according to the desired penetration depth of the instrument into the bone tunnel, and the perforations along the length of the instrument are configured to distribute a fluidic agent into the bone tunnel. Additional features such as optical emitters including, but not limited to, LEDs of the visible, ultraviolet, or infrared wavelengths, thermal or electrical conductors, or additional chemical diffusion agents either coating or extrinsically introduced through the instrument may also be integrated in order to achieve its function to induce the fluidic agent to undergo phase transition whether by polymerization, setting, or curing. It is important to note that the injector need not be rigid and may be flexible and capable of being manipulated to facilitate insertion into the bone tunnel.

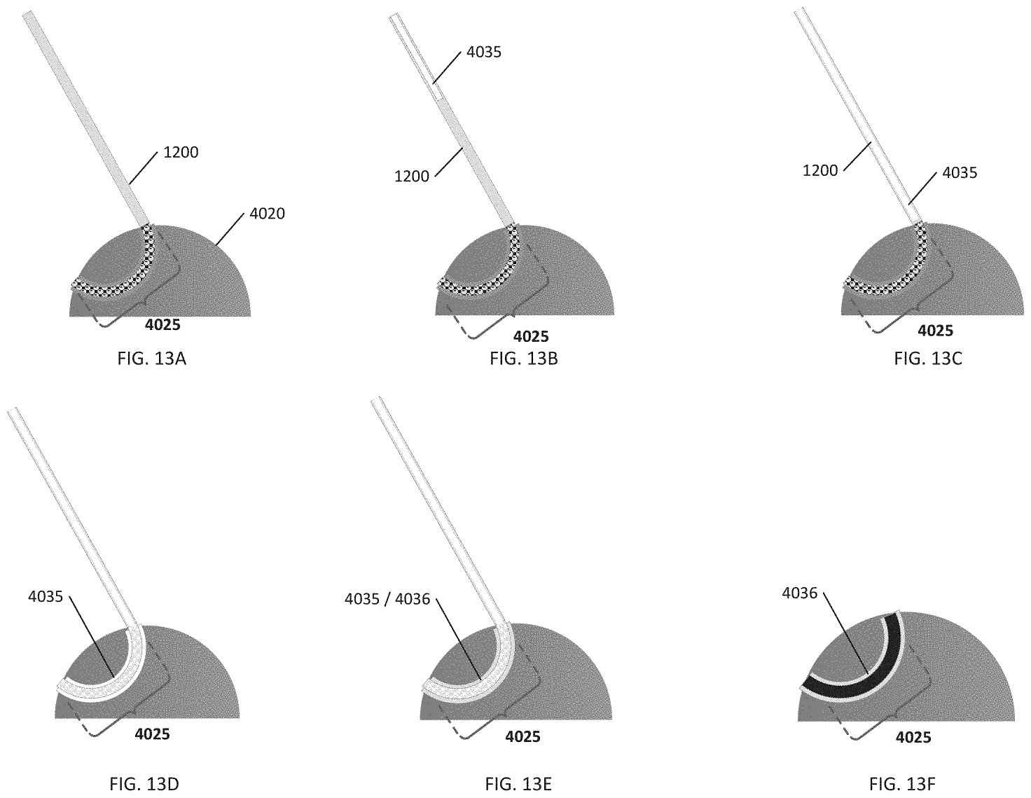

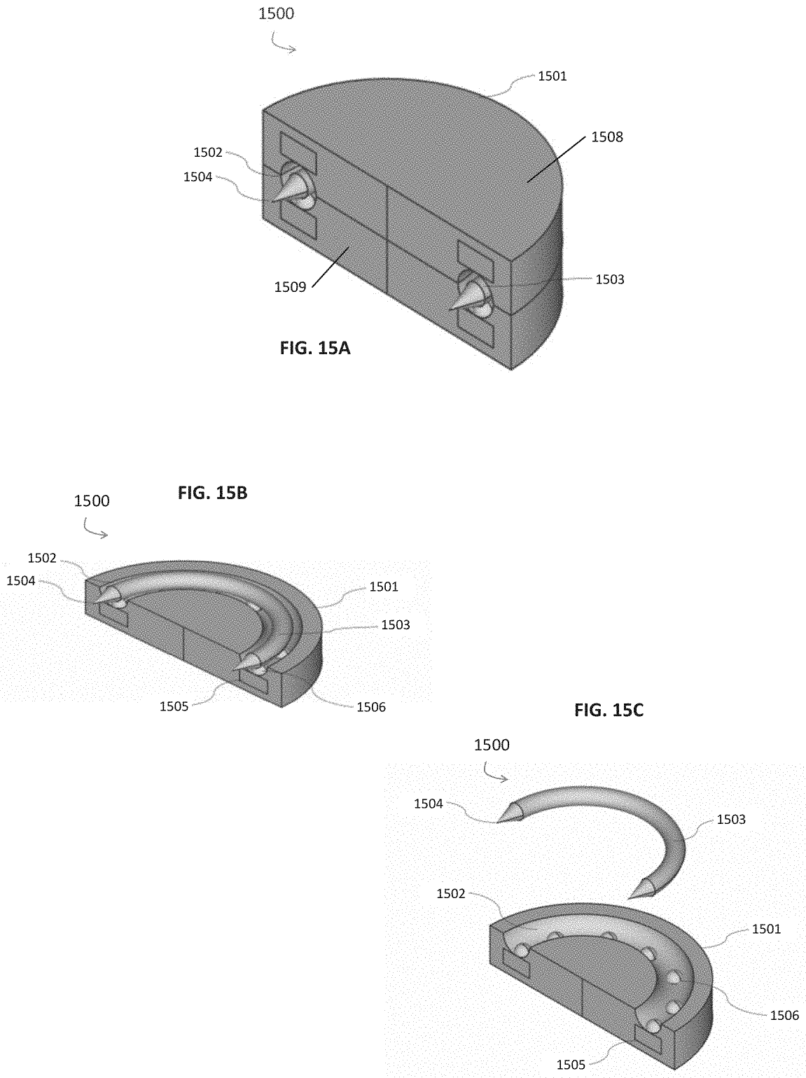

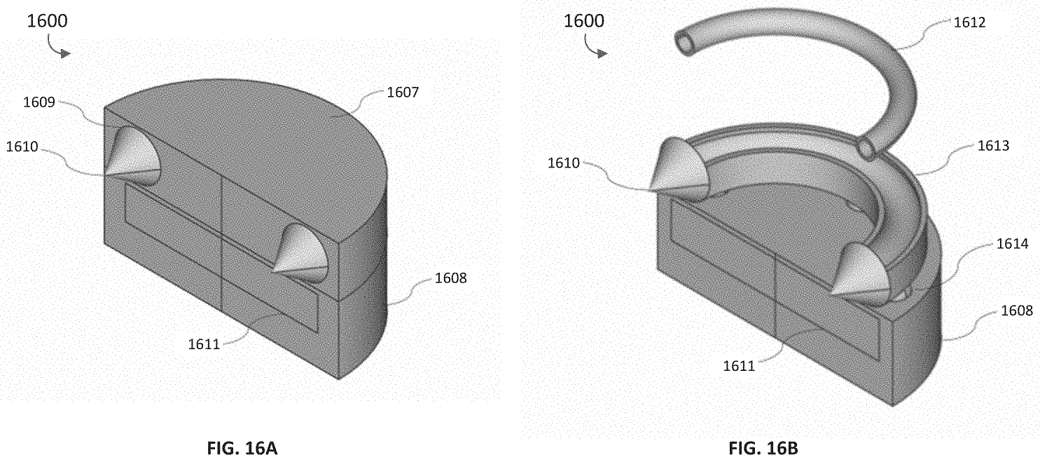

FIGS. 13A to 13F depict partial cross-sectional views of curved tubular injector 1200, like those described with respect to FIGS. 12A and 12B, in various stages of operation, wherein curved tubular injector 1200 is configured to generate an implanted bracing apparatus in situ. FIG. 13A shows the step of introducing curved tubular injector 1200 into bone tunnel 4025 within bone 4020. Bone tunnel 4025 can be created prior to introduction of curved tubular injector 1200 through, for example, the methods and devices described herein with respect to FIGS. 15A, 15B, 15C, 16A, and 16B. According to some embodiments, a single tubular arm, either flexible or rigid, can be used to maneuver injector 1200 into bone tunnel 4025. Curved tubular injector 1200, whether flexible or rigid, can be inserted into one end of bone tunnel 4025 (as shown in FIGS. 13A to 13F) or, in the alternative, into both ends, in which case a first end and a second end are connected by bone tunnel 4025. According to one aspect of the embodiments, bone tunnel 4025 is configured to receive fluidic agent 4035. In some embodiments, curved tubular injector 1200 can be part of a motorized assembly, such as that of the tunneling devices described with respect to FIGS. 15A, 15B, 15C, 16A, and 16B.

FIGS. 13B, 13C, and 13D show the steps of injecting the natural or synthetic fluidic agent 4035, as it moves from injector 1200 into bone tunnel 4025. According to one aspect of the embodiments, a predetermined amount of fluidic agent 4035, based on volume needed to fill the bone tunnel, can either be manually injected (such as using a syringe or similar instrument) or automatically injected (such as with a motorized pump).

FIG. 13E shows the natural or synthetic fluidic agent 4035 in the process of spontaneously undergoing, or being activated/induced to undergo, phase transition into a solid bracing apparatus 4036 inside bone tunnel 4025. The nature of this phase transition will depend on the selected fluidic agent 4035 being used. Example embodiments of the fluidic agent can be one or more of a self-assembling polymer, a UV-cured resin, a thermosetting polymer, a chemically-cured material, a chemically-crosslinked material, among others. As shown in FIG. 13F, the curved tubular injector 1200 has been retracted from bone tunnel 4025, leaving behind the newly generated bracing structure 4036 after having either partially or fully undergone phase transition into a solid material.

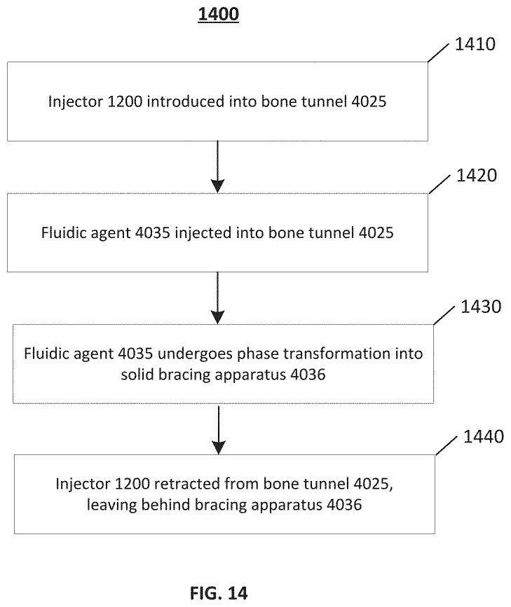

FIG. 14 is a flow diagram depicting an example embodiment of a method 1400 for generating bracing apparatus 4036 in situ, in accordance with the embodiments described with respect to FIGS. 12A to 12B and 13A to 13F.

At Step 1410, an injector 1200 is introduced into bone tunnel 4025. According to some embodiments, injector 1200 can comprise a curved tubular injector having a single tubular arm that is either flexible or rigid. Bone tunnel 4025 can comprise a cavity inside bone that can include one or more openings at the bone surface. Injector 1200 can be inserted into one end of bone tunnel 4025 or, according to some embodiments, into both ends of bone tunnel 4025. In some embodiments, injector 1200 can be part of a motorized assembly, such as the tunneling devices described herein with respect to FIGS. 15A, 15B, 15C, 16A, and 16B.

At Step 1420, fluidic agent 4035 is injected into bone tunnel 4025. Example embodiments of the fluidic agent can be one or more of a self-assembling polymer, a UV-cured resin, a thermosetting polymer, a chemically-cured material, a chemically-crosslinked material, among others. According to one aspect of the embodiments, a predetermined amount of fluidic agent 4035, based on volume needed to fill the bone tunnel, can either be manually injected (such as by using a syringe or similar instrument) or automatically injected (such as with a motorized pump).

At Step 1430, fluidic agent 4035 undergoes phase transition from fluid 4035 into solid bracing apparatus 4036 inside bone tunnel 4025. The nature of the phase transition depends on the selected fluidic agent 4035 being used.

At Step 1440, injector 1200 is retracted or withdrawn from bone tunnel 4025, leaving behind the newly generated bracing apparatus 4036 after having either partially or fully undergone phase transition into a solid material.

### Example Embodiments of Tunneling Devices and Methods Relating Thereto

Example embodiments of tunneling devices for creating a bone tunnel, and methods relating thereto, will now be described.

FIG. 15A is a perspective view depicting an example embodiment of a tunneling device 1500 for creating one or more tunnels in a bone material, which can be used with any of the previously described embodiments. According to some embodiments, tunneling device 1500 includes a housing 1508 comprising at least one surface 1509 configured to interface with a bone material, a channel 1502 disposed within housing 1508, and a curved impactor 1503 configured to travel along channel 1502 at high speeds. Channel 1502 is shown to be circular in cross-section, but those of skill in the art will appreciate that the cross-sectional shape of channel 1502 and impactor 1503, configured to travel therein, may be of any geometry. According to some embodiments, the path of channel 1502 within tunneling device 1500 can have an arcuate geometry. Furthermore, as seen in FIG. 15A, tunneling device 1500, including housing 1508 and channel 1502 disposed therein, comprises a semi-circular shape subtending an angle of 180 degrees. Those of skill in the art, however, will appreciate that tunneling device 1500, housing 1508, or channel 1502, may comprise a semi-circular shape subtending an angle of more or less than 180 degrees, or may have a non-circular shape. In addition, the length of curved impactor 1503 may be either greater than or less than the length of channel 1502 of tunneling device 1500.

According to another aspect of the embodiments, impactor 1503 includes one or more pointed ends 1504, as seen in FIG. 15A, which are configured such that the energy upon striking a bone material can cause a bone tunnel to lengthen. In some embodiments, an internal propulsion mechanism 1501 transfers energy to impactor 1503 to generate a back-and-forth motion within channel 1502. Internal propulsion mechanism 1501 can comprise any technology used to generate motion including, but not limited to, one or more of a piezoelectric motor, an electrical induction motor, magnetic propulsion, pneumatic propulsion, hydraulic propulsion, mechanical (e.g., linkages, gears, etc.), or any combination thereof.

FIGS. 15B and 15C depict a perspective partial cross-sectional view and a perspective partial exploded view, respectively, of an example embodiment of tunneling device 1500 comprising a piezoelectric motor 1501 configured to move impactor 1503. According to one aspect of the embodiments, piezoelectric motor 1501 can comprise a plurality of piezoceramic elements 1505 disposed along the length of channel 1502, wherein piezoceramic elements 1505 are configured to expand and contract in response to electrical energy and actuate one or more bumpers 1506. According to another aspect of the embodiments, in response to being actuated by piezoceramic elements 1505, the one or more bumpers 1506 are then configured to exert one or more forces on impactor 1503, which can cause impactor 1503 to move according to linear or circular motions, thereby moving impactor 1503 along the path of channel 1502 according to a predetermined back-and-forth motion. The repeated impact from impactor 1503, whose trajectory is defined by impactor 1503 travelling along path of channel 1502, then results in a curved bone tunnel.

As best seen in FIGS. 15B and 15C, according to some embodiments, impactor 1503 can comprise a solid apparatus including one or more solid conical tips 1504 on each end. Those of skill in the art, however, will appreciate that impactor 1503 can include one or more tips 1504 having a different geometry including, but not limited to, a hollow cylinder, hemisphere, or truncated cone, along with other tip features, such as flutes. Additionally, certain embodiments of tunneling device 1500 may use any of the implantable bracing apparatuses described herein, or any components thereof, such as those described with respect to FIGS. 1A, 1B, 2A, 2B, 3A, 3B, 4A, 4B, 5A, 5B, 6A, 6B, 7A, 7B, 8, 9, 10A, 10B, 11A, 11B, 11C, or 11D, as the impactor to form a curved bone tunnel, such that the bracing apparatus may be left within the tunnel after the tunnel is formed. Additionally, according to some embodiments, piezomotor 1501 can comprise one or more of a piezo inertia motor, piezo ultrasonic resonance motor, or piezo walk motor. Those of skill in the art will recognize that the motion of piezo actuation can include stacking, tubing, expanding, shearing, walking, bending, bimorph flexing, and bimorph bending.

FIGS. 16A and 16B depict a perspective view and a perspective exploded view, respectively, of another embodiment of tunneling device 1600. According to one aspect of the embodiments, tunneling device 1600 can comprise a first subassembly 1607 and a second subassembly 1608 configured to couple with first subassembly 1607, wherein a curved channel 1609 and impactor 1613 are disposed in first subassembly 1607 of tunneling device 1600, and a piezoelectric motor 1611 is disposed in second subassembly 1608 of tunneling device 1600. Although the cross-section of channel 1609 is shown to be circular in FIG. 16A, those of skill in the art will appreciate that the channel cross-section may be of any geometry. In addition, although impactor 1613 is depicted as a hollow curved cylinder with a plurality of pointed conical tips 1610, those of skill in the art will recognize that impactor 1613 and tips 1610 can comprise the same or similar configurations as those described above with respect to FIGS. 15A, 15B, and 15C.

According to another aspect of the embodiments, a piezoelectric motor 1611 disposed in subassembly 1608 of tunneling device 1600 is configured to actuate a motion to drive impactor 1613 in a back-and-forth motion to create a curved bone tunnel. In some embodiments, impactor 1613 can comprise a needle having “pointy” cone-shaped tips 1610 for impaction drilling. The repeated impact from impactor 1613, whose trajectory is defined by impactor 1613 travelling along path of channel 1609, then results in a curved bone tunnel.

According to another aspect of the embodiments, after impactor 1613 is propelled by bumpers 1614 actuated by piezoelectric motor 1611 to create a bone tunnel, an implantable bracing apparatus 1612, such as any of the embodiments described herein with respect to FIGS. 1A, 1B, 2A, 2B, 3A, 3B, 4A, 4B, 5A, 5B, 6A, 6B, 7A, 7B, 8, 9, 10A, 10B, 11A, 11B, 11C, or 11D, is subsequently inserted into the bone tunnel by tunneling device 1600. Bracing apparatus 1612 can be contained in a holding space within the impactor 1613, and deployed after the removal of impactor tips 1610. Bracing apparatus 1612 is then left to remain in the bone tunnel.

In many of the embodiments disclosed herein, tunneling devices 1500 and 1600 can also incorporate a feed mechanism (not shown) to insert a suture through the bracing apparatus that is situated in the bone tunnel. Because the bone tunnel and bracing can be circular arcs, a suture can easily pass through without the need for specialized hooks or pincers to pull the suture from the exit hole after a suture has been introduced through the entrance hole.

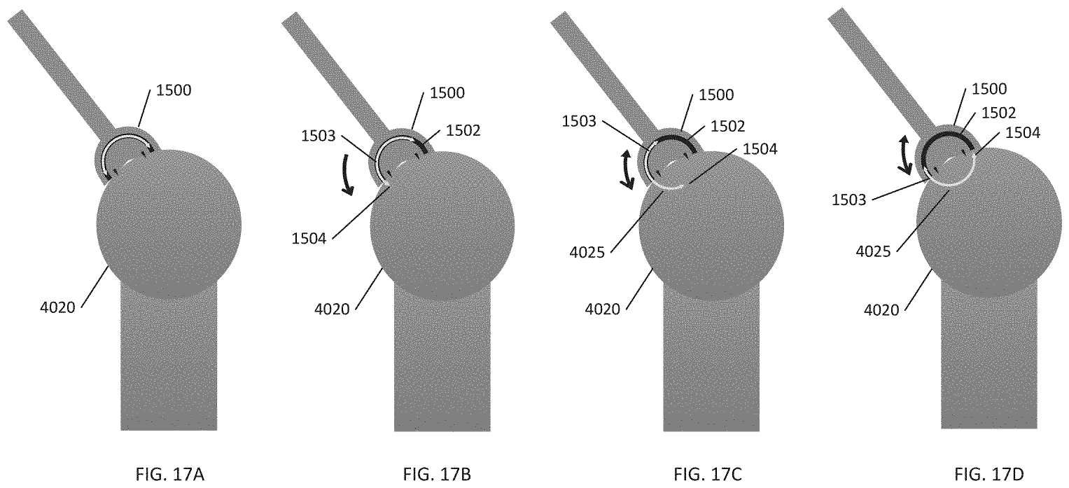

FIGS. 17A to 17D are progressive diagrammatic views of an example embodiment of a tunneling device 1500 in various stages of operation, wherein tunneling device 1500 is configured to create a bone tunnel 4025 in a unidirectional manner. FIG. 17A depicts an initial stage wherein tunneling device 1500 is placed against the surface of bone 4020 at a predetermined location identified as the tunneling site. According to one aspect of the embodiments, tunneling device 1500 is held stationary against the predetermined location of bone 4020 during the process in which a bone tunnel is created.

FIG. 17B depicts a subsequent stage in which impactor 1503 is actuated, and travels along channel 1502 of tunneling device 1500, which can cause the pointed tip 1504 of impactor 1503 to make contact with the surface of bone 4020, as indicated by the arrow. As described above, impactor 1503 can be actuated using a piezomotor (not shown), such as, for example, a piezo inertia motor, piezo ultrasonic resonance motor, piezo walk motor, or any similar mechanism to cause the impactor 1503 to travel along channel 1502 at a high speed.

FIG. 17C depicts a stage of operation in which a continuous back-and-forth actuation (as indicated by the bi-directional arrow) of impactor 1503 along channel 1502 has caused pointed tip 1504 of impactor 1503 to further progress into bone 4020, thereby creating a partial bone tunnel 4025.

FIG. 17D depicts a near-final stage of operation in which the continuous back-and-forth actuation of impactor 1503 (as indicated by the bi-directional arrow) along channel 1502 has caused pointed tip 1504 of impactor 1503 to further progress into bone 4020, thereby completing the bone tunnel 4025. As can be seen in FIG. 17D, the completed bone tunnel 4025 can comprise a first opening created by the entry of pointed tip 1504 (FIG. 17B) and a second opening created by the exit of pointed tip 1504 (FIG. 17D). According to another aspect of the embodiments, impactor 1503, having a curved or arcuate body, is configured to create bone tunnel 4025, which can similarly have a curved or arcuate shape.

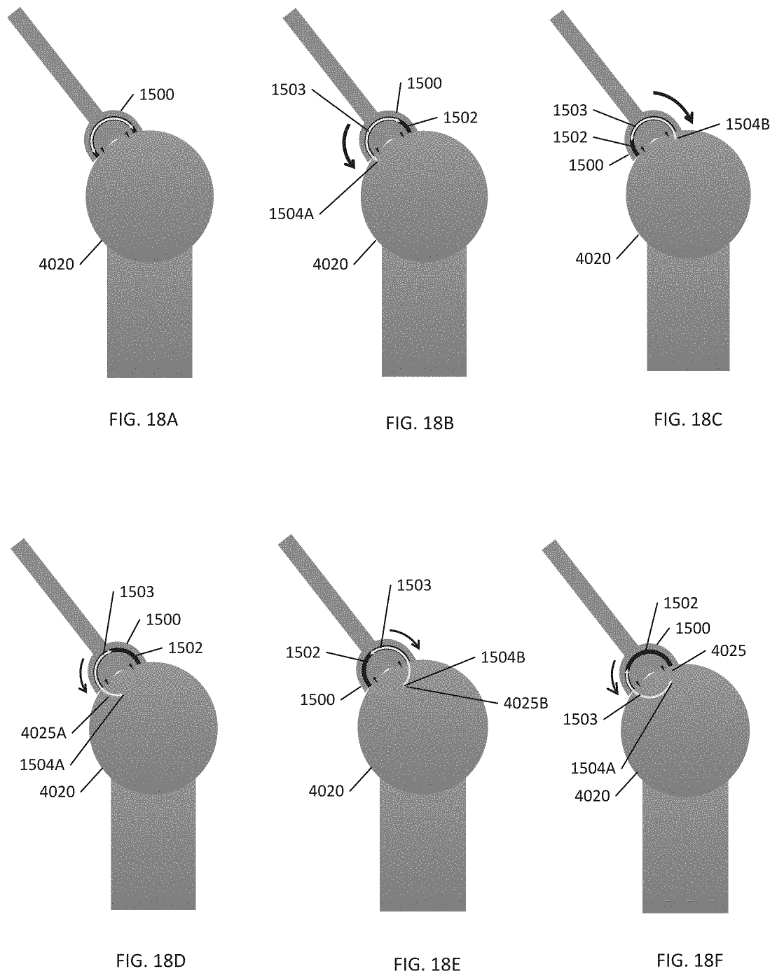

FIGS. 18A to 18F are progressive diagrammatic views of another example embodiment of a tunneling device 1500 in various stages of operation, wherein tunneling device 1500 is configured to create a bone tunnel 4025 in a bi-directional manner.

FIG. 18A depicts an initial stage wherein tunneling device 1500 is placed against the surface of bone 4020 at a predetermined location identified as the tunneling site. According to one aspect of the embodiments, tunneling device 1500 is held stationary against the predetermined location of bone 4020 during the process in which a bone tunnel is created.

FIG. 18B depicts a subsequent stage in which impactor 1503 is actuated and travels along channel 1502 of tunneling device 1500 in a counter-clockwise direction (as indicated by the arrow), which can cause a first pointed tip 1504A of impactor 1503 to make contact at a first entry point on the surface of bone 4020. As described above, impactor 1503 can be actuated using a piezomotor (not shown), such as, for example, a piezo inertia motor, piezo ultrasonic resonance motor, piezo walk motor, or any similar mechanism to cause the impactor 1503 to travel along channel 1502 at a high speed in a back-and-forth motion.

FIG. 18C depicts a subsequent stage in which impactor 1503 travels along channel 1502 of tunneling device 1500 in a clockwise direction (as indicated by the arrow), which can cause a second pointed tip 1504B of impactor 1503 to make contact at a second entry point on the surface of bone 4020.

FIG. 18D depicts a subsequent stage of operation in which impactor 1503 travels along channel of 1502 of tunneling device 1500 again in a counter-clockwise direction (as indicated by the arrow), causing first pointed tip 1504A of impactor 1503 to further progress into bone 4020 through the first entry point, thereby creating a first partial bone tunnel 4025A.

FIG. 18E depicts a subsequent stage of operation in which impactor 1503 travels along channel 1502 of tunneling device 1500 again in a clockwise direction (as indicated by the arrow) causing second pointed tip 1504B of impactor 1503 to further progress into bone 4020 through the second entry point, thereby creating a second partial bone tunnel 4025B.

FIG. 18F depicts a near-final stage of operation in which the continuous back-and-forth actuation of impactor 1503 along channel 1502 has caused first pointed tip 1504A of impactor 1503 to further progress into bone 4020 through the first entry point, thereby connecting the two partial bone tunnels (4025A, 4025B) to form a completed bone tunnel 4025. As can be seen in FIG. 18F, the completed bone tunnel 4025 can comprise a first opening created by the entry of first pointed tip 1504A and a second opening created by the entry of second pointed tip 1504B. According to another aspect of the embodiments, impactor 1503, having a curved or arcuate body, is configured to create bone tunnel 4025, which can similarly have a curved or arcuate shape.

Although FIGS. 17A to 17D and FIGS. 18A to 18F are shown with tunneling device 1500, those of skill in the art will understand that the methods described herein can be utilized with any of the disclosed tunneling devices, including the embodiments described with respect to FIGS. 15A, 15B, 15C, 16A, and 16D.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. An implantable bracing apparatus for supporting a suture, the apparatus comprising a tube comprising a length that is curved, partially curved, or straight, and at least a portion of which is configured to be implanted within a bone tunnel, the tube comprising:
an outer surface configured to interface with the bone tunnel;
an inner lumen configured to pass one or more sutures therethrough and to prevent the one or more sutures from contacting at least a portion of the bone tunnel;
a first open end corresponding with a first entry point of the bone tunnel; and
a second open end corresponding with a second entry point of the bone tunnel.

2-3. (canceled)

4. The apparatus of claim 1, wherein the outer surface includes one or more of a plurality of grooves or a plurality of fins.

5. The apparatus of claim 1, wherein the tube comprises a metallic material, a natural material, a synthetic material, an organic material, an inorganic material, a biodegradable polymer, a non-biodegradable polymer, or a combination thereof.

6-7. (canceled)

8. The apparatus of claim 1, wherein one or both of the inner lumen and outer surface comprise at least one of an osteoconductive material, a protein, a biologic, or a synthetic molecule configured to promote osseointegration and bone ingrowth.

9. The apparatus of claim 8, wherein the osteoconductive material comprises hydroxyapatite.

10. The apparatus of claim 1, wherein at least a portion of the tube comprises a nanotextured surface.

11. The apparatus of claim 1, further comprising a plurality of end cap accessories configured to mate with the first open end and the second open end.

12. The apparatus of claim 11, wherein at least one of the plurality of end cap accessories comprises one or more of a flanged head, a through hole configured to enable passage of the one or more sutures, and an eyelet.

13. The apparatus of claim 11, wherein at least one of the plurality of end cap accessories comprises a ridged feature configured to lock the at least one of the plurality of end cap accessories with a corresponding ridge locking feature disposed on at least one of the first open end or the second open end.

14. The apparatus of claim 1, further comprising one or more screw caps, and wherein one or both of the first open end and the second open end comprises a threaded portion configured to receive the one or more screw caps.

15-30. (canceled)

31. An implantable bracing apparatus for supporting one or more sutures, the apparatus comprising a cylindrical volume comprising one or more helices at least a portion of which is configured to be implanted within a bone tunnel, the cylindrical volume comprising:
an outer portion of the cylindrical volume configured to interface with the bone tunnel;
an inner portion of the cylindrical volume configured to pass suture(s) therethrough and to prevent the one or more sutures from contacting at least a portion of the bone tunnel;
a first open end corresponding with a first entry point of the bone tunnel; and
a second open end corresponding with a second entry point of the bone tunnel.

32. The apparatus of claim 31, wherein the cylindrical volume further comprises a single helical coil.

33. The apparatus of claim 31, wherein the cylindrical volume further comprises a double helix.

34. The apparatus of claim 31, wherein the cylindrical volume further comprises four helical coils.

35. The apparatus of claim 31, wherein the cylindrical volume further comprises a latticework of struts.

36. The apparatus of claim 35, wherein the latticework of struts comprises one or more of a plurality of triangular cells, a plurality of quadrilateral cells, a plurality of hexagonal cells, a plurality of octagonal cells, or a combination thereof.

37-74. (canceled)
